(19) United States
(12) Patent Application Publication
(10) Pub. No.: US 2006/0141421 A1
(43) Pub. Date: Jun. 29, 2006

(54) System and method for detecting substances related to oral health

(75) Inventors: Laura F. Braunecker, Alpharetta, GA (US); Eric F. Wagner, Sherwood, WI (US); John Gavin MacDonald, Decatur, GA (US)

Correspondence Address: SCHWEGMAN, LUNDBERG, WOESSNER & KLUTH, P.A., P.O. BOX 2938, MINNEAPOLIS, MN, 55402, US

(73) Assignee: Kimberly-Clark Worldwide, Inc.

(21) Appl. No.: 11/027,667

(22) Filed: Dec. 28, 2004

(51) Int. Cl. A61K 7/16

(52) U.S. Cl. 433/215

(57) ABSTRACT

The present invention relates to a low cost and portable system and method for detecting substances related to oral health. In one example embodiment, the system includes a sensor that is incorporated into an oral healthcare device (e.g., a toothbrush). The sensor detects bacterial and/or chemical levels that are within a mouth in order to provide an indication as to the presence of oral malodor and/or bacteria within the mouth.

### FIELD OF THE INVENTION

The present invention relates to the detection of substances that influence oral health, and more specifically to a system and method that detect the presence of bacteria and other substances which may cause halitosis, plaque, tartar, gingivitis, and periodontal disease.

### BACKGROUND OF THE INVENTION

People are continually trying to monitor their oral health because of concerns relating to breath odor and disease. The desire to reduce breath odor and disease has made developing convenient, accurate, and reliable devices for providing oral healthcare a goal of healthcare professionals and researchers.

The ability to reliably monitor the presence of substances that cause halitosis, gingivitis, plaque, tartar, cancer and viral infections (e.g., herpes simplex) is a key element in preventing unpleasant, uncomfortable, or disabling oral heath ailments. As an example, halitosis alone affects the quality of life of millions of people each year thereby contributing to embarrassing encounters and psychological stress. In addition, the bacterium that causes halitosis can lead to unsightly tartar, inflammation of the gums and even periodontal disease when such bacterium goes undetected and untreated.

Historically, people have visited their dentist once or twice a year in order to learn about the effectiveness of their dental hygiene habits. Unfortunately, these periodic visits may not happen often enough to accurately indicate the presence of harmful substances in the mouth.

May people also rely on mouth treatments (e.g., mouthwashes and/or breath mints) to improve their breath and reduce the amount of harmful substances in the mouth. One drawback with these types of treatments is that they often mask the origin of a problem (i.e., the presence of damaging bacteria) without informing the user that there is actually a problem.

One known device for detecting oral bacteria or other harmful substances is a halitosis detector device (see, e.g., U.S. Pat. No. 4,823,803). Most halitosis detector devices typically include a chamber that receives exhalation to be tested from a tube. A sensor, which detects malodorant gases of predetermined chemical compositions, is typically located in the chamber to detect the concentration of the malodorant gases within the exhalation. One drawback with existing halitosis detector devices is that they are used separately from normal dental activity thereby making such devices cumbersome and impractical for daily use.

### SUMMARY OF THE INVENTION

The present invention provides a low-cost and/or disposable system and method for detecting substances that influence oral health. The system and method are simple to use and may incorporate a commonly used oral implement.

In some forms, the system includes a body having a handle and a head that is attached to the handle. The system further includes a sensor that is attached to the body. The sensor is adapted to detect a substance that influences oral health when the system is placed in (or near) a mouth.

In other forms, the system includes a finger glove and a sensor that is attached to the finger glove. The sensor is adapted to detect a substance (e.g., bacteria) that influences oral health when the system is placed in a mouth.

In still other forms, the system includes a dental implement (e.g., a mouthpiece) and a sensor that is attached to the dental implement. The sensor is adapted to detect a substance that influences oral health when the system is placed within a mouth.

In some forms, the method includes placing a system near a mouth where the system includes a body having a handle and a head that is attached to the handle. The system further includes a sensor that is attached to the body. The method further includes detecting the presence of a substance that influences oral health using the system.

In other forms, the method includes placing a system near a mouth where the system includes a finger glove and a sensor that is attached to the finger glove. The method further includes detecting the presence of a substance that influences oral health using the system (e.g., by inserting the finger glove into the mouth).

In still other forms, the method includes placing a system near a mouth where the system includes a dental implement (e.g., a mouthpiece) and a sensor that is attached to the dental implement. The method further includes detecting the presence of a substance (e.g., bacteria) that influences oral health using the system.

### BRIEF DESCRIPTION OF THE DRAWINGS

The above-mentioned and other features will become more apparent and better understood by reference to the following description of the invention taken in conjunction with the accompanying drawings.

FIG. 1 illustrates an example system that may be used to detect substances that influence oral health.

FIG. 2 illustrates the system of FIG. 1 when the system is placed near a mouth.

FIG. 3 illustrates example electronic circuitry that may be incorporated into a system of the present invention.

FIG. 4 illustrates another example embodiment of the system shown in FIG. 1.

FIG. 5 illustrates another example embodiment of the system shown in FIG. 1.

FIG. 6 illustrates an example method of detecting substances that influence oral health.

FIG. 7 illustrates another example system that may be used to detect substances which influence oral health.

FIG. 8 illustrates another example method of detecting substances that influence oral health.

FIG. 9 illustrates another example system that may be used to detect substances that influence oral health.

FIG. 10 illustrates another example method of detecting substances which influence oral health.

### DESCRIPTION OF THE INVENTION

The system and method of the present invention may provide an efficient, healthy and low-cost way to detect substances that influence oral health. The system and method may incorporate a commonly used oral device such that the system and method are easy to use.

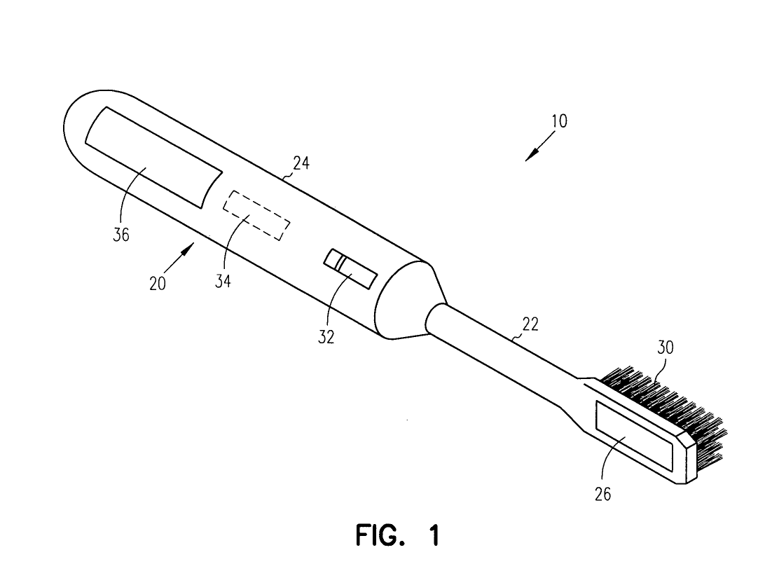

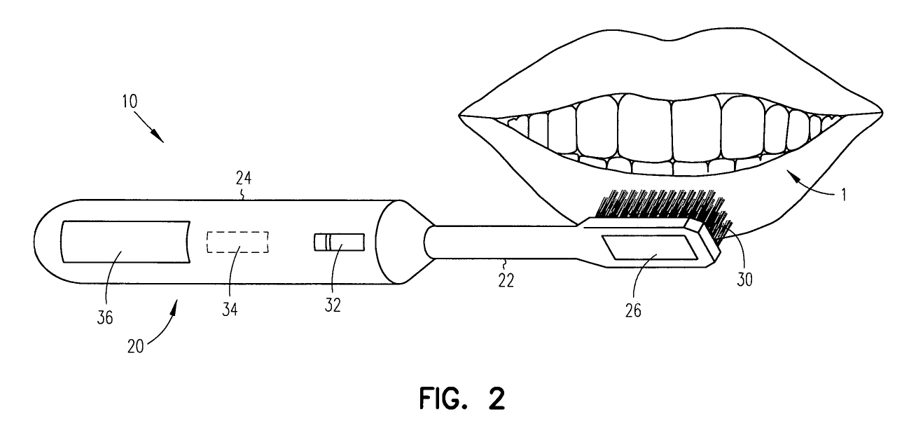

FIG. 1 illustrates an example system 10 of the present invention. The system 10 includes a body 20 having a head 22 and a handle 24. The system further includes a sensor 26 that is attached to the body 20. As shown in FIG. 2, the sensor 26 is adapted to detect substances that influence oral health when the system 10 is placed near a mouth 1.

In the example embodiment illustrated in FIG. 1, the system 10 is a toothbrush that includes a set of bristles 30 for brushing teeth. It should be noted that although system 10 is shown as being a toothbrush, system 10 may be any type of oral care device.

The system 10 further includes a switch 32, a power source 34, and a display 36 that are connected to the sensor 26 within the body 20 of system 10. In some embodiments, the head 22 and/or bristles 30 may be detachable from the handle 24 and/or movable relative to the rest of the body 20.

Sensor 26 may be a polymer that is designed to collect specific molecules related to oral health. The sensor 26 may be integrated into the head 22 (or handle 24) such that the sensor 26 is exposed to a user's saliva and/or breath upon brushing. In some embodiments, the bristles 30 may convey saliva to the sensor 26.

It should be noted that sensor 26 may be any type of sensor that is known now or discovered in the future. The type of sensor 26 that is used the system 20 will depend in part on the application where the system 10 is to be used.

The display 36 may be an LCD, LED or any other type of display. The display 36 may be integrated into the handle 24 (or head 22) such that a user is able to view information which appears on the display 36.

The switch 32 may be a bipolar switch with an “on” position and an “off” position. The switch 32 may be integrated into the body 20 such that a user is able to operate the system 20 during (or after) brushing.

Power source 34 may be a single-use battery that requires replacement after some length of time, or a rechargeable battery that the user recharges by placing the system 10 in a charging cradle. In addition, power source 34 may be incorporated into the body 20 such that the power source 34 can not be seen but is easily replaced or recharged.

A user operates the system 10 by manipulating the switch 32 to an “on” position. The user brushes his or her teeth such that the sensor 26 collects molecules related to oral malodor (e.g., hydrogen sulfide, dimethyl sulfide, methyl mercaptan, and ethyl mercaptan) and/or harmful bacteria (e.g., gingivitis bacteria). The sensor 26 provides information to the display 36 regarding chemical and bacterial levels that are present in the user's mouth or on the user's breath.

This information may indicate the presence of oral malodor, plaque or tartar-causing substances, or bacteria that causes gingivitis and/or periodontal disease. In some embodiments, the information may also indicate the presence of cancer or viral infections (e.g., herpes simplex). It should be noted that the sensor 26 may detect relative concentrations of one or more substances.

As an example, display 36 may indicate a “malodor scale” that ranges from one to ten based on the sulfur level detected on the user's breath. A malodor level of one may indicate acceptable breath while a level of ten may indicate unacceptable breath.

As another example, display 36 may indicate a “bacterial scale” that ranges from one to ten. A bacterial level of one may indicate that there is a minimum of harmful gingivitis bacteria which are present in the mouth while a level of ten may indicate that there is an unhealthy level of harmful gingivitis bacteria in the mouth.

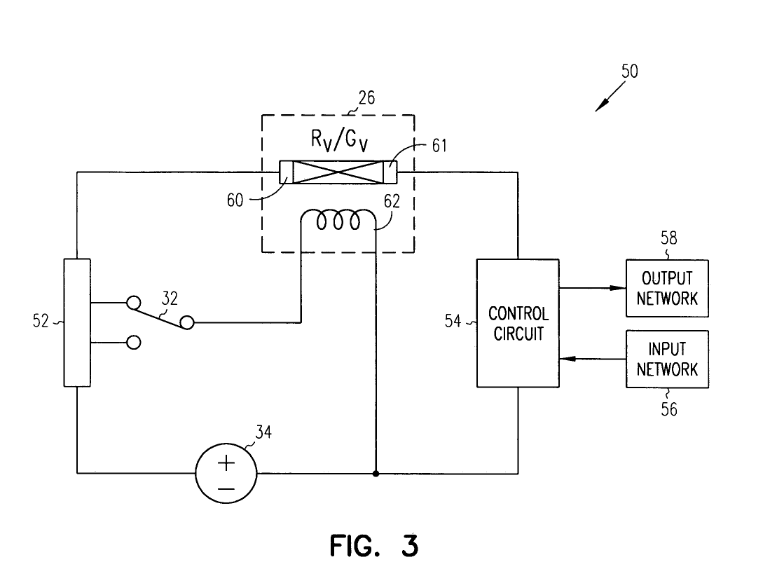

As shown in FIG. 3, sensor 26, switch 32 and power source 34 may be incorporated into electronic circuitry 50. In the illustrated example embodiment, electronic circuitry 50 includes a constant voltage generator circuit 52, a control circuit 54, an input network 56 and an output network 58.

In some embodiments, sensor 26 includes a first electrode 60, a second electrode 61, and a heating element 62. Sensor 26 may be sensitive to anion-adsorptive reducing gases of prescribed chemical compositions such that sensor 26 has a variable resistance R or conductance G when the sensor 26 is thermally activated. As an example, sensor 26 may be sensitive to chemicals that are normally associated with oral malodor (e.g., hydrogen sulfide, dimethyl sulfide, methyl mercaptan, and ethyl mercaptan).

Heating element 62 is connected across power source 34 while sensor 26 is connected across power source 34 through control circuit 54. The control circuit 54 is responsive to a variable differential voltage produced between first electrode 60 and second electrode 61 as the resistance R or conductance G varies across sensor 26.

The control circuit 54 is coupled to an input network 64 that supplies the control circuit 54 with data signals during operation of the system 10. The control circuit 54 is also coupled to an output network 66 that receives signals from the control circuit 54 and then delivers output signals to the display 36.

During operation of the system 10, sensor 26 is thermally activated using the heating element 62. The system 10 is then exposed to malodorant gases. The resistance R or conductance G between the first electrode 60 and the second electrode 61 vary in proportion to the concentration of the malodorant substances. Therefore, a signal voltage, which is in proportion to the concentration of the detected malodorant gases, is produced between the first electrode 60 and the second electrode 61. The signal voltage is then supplied to control circuit 54.

In some embodiments, constant voltage generator circuit 52 applies a voltage to the heating element 62 when the switch 32 is in the “on” position for the detection of malodorant gases. In addition, constant voltage generator circuit 52 may also apply a voltage to the heating element 62 when the switch is in the “off” position in order to regenerate the sensor 26. The “off” voltage may be much higher than the “on” voltage such that the heating element 62 burns away the molecules that were collected by the sensor 26.

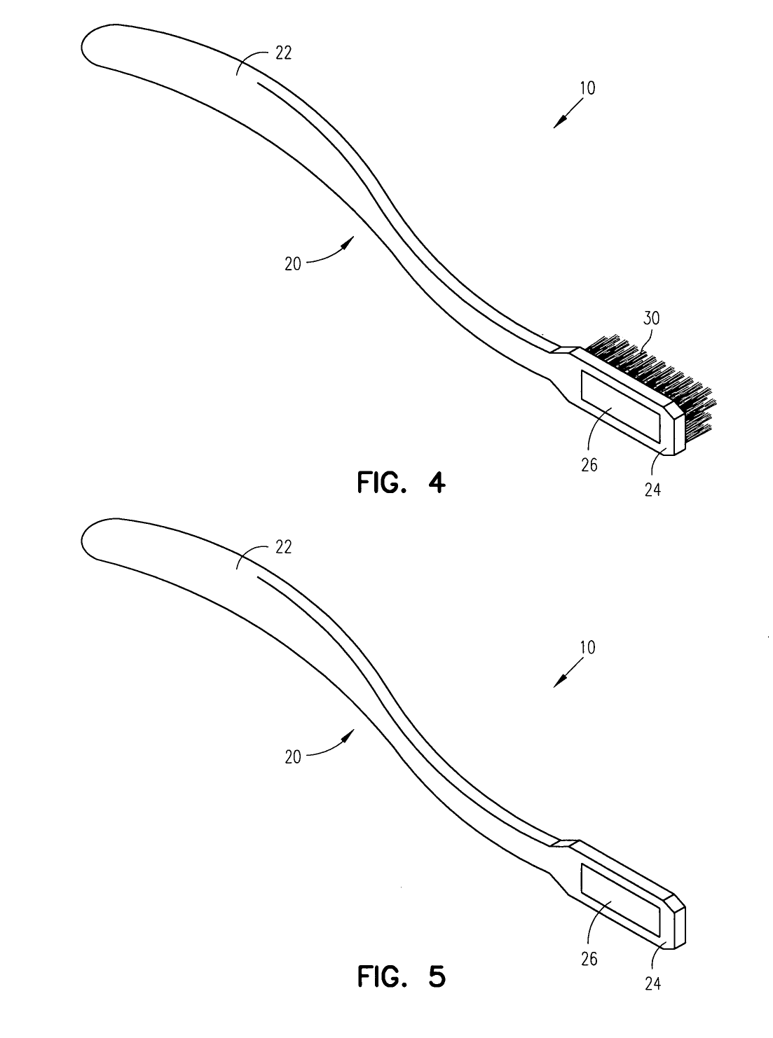

FIG. 4 shows an alternative embodiment of the system 10. In the illustrated embodiment, the sensor 26 may be a polymer that is designed to collect specific molecules related to oral health and then change color as target molecules are collected. In some forms, the intensity of the color is proportional to the number of target molecules that are collected.

As an example, sensor 26 may be designed to collect molecules related to oral malodor (e.g., sulfur). The sensor 26 may transition from white to red as the sensor 26 collects the relevant molecules. When the sensor 26 detects more relevant molecules, the intensity of the red color increases to provide a user with a relative measure as to the extent of the oral malodor.

It should be noted that the color change within the sensor 26 may indicate relative chemical and/or bacterial levels in a user's mouth or on the user's breath. In some embodiments, an individual may be able to compare the color of the sensor 26 to a printed color chart (e.g., printed on the handle of the toothbrush) such that the user is able to determine the levels of substances which are present in the mouth.

As an example, sensor 26 may indicate a scale that ranges from light pink to deep red based on the sulfur level detected on the user's breath. A light pink color indicates acceptable breath and a deep red color indicates unacceptable breath.

As another example, sensor 26 may indicate a scale that ranges from light blue to deep blue. A light blue color may indicate that an acceptable level of harmful gingivitis bacteria is present in the mouth and a deep blue may indicate that an unhealthy level of harmful gingivitis bacteria is present in the mouth.

In some embodiments, sensor 26 may completely release the molecules that were collected during brushing such that the sensor 26 returns to its original color. The molecules may be released from the sensor 26 over time. The amount of time that it takes for the sensor to regenerate will depend in part on the bacterial and/or chemical levels that were detected by sensor 26.

Once the sensor 26 has regenerated, a user may be able to use the system 10 as described above in order to detect the presence of undesirable substances in the mouth. In other embodiments, a portion of the system 10 may be stored or treated in water (or some other liquid) that actively removes the malodorant substances which are collected by the sensor 26.

Another example embodiment of the system 10 is shown in FIG. 5 where the system 10 is operated solely as a portable oral malodor detector without the need to brush teeth (i.e., the system has a toothbrush type configuration without the bristles). Individuals may use the system 10 by exhaling a fixed number of times (e.g., three times) on the sensor 26 and/or by engaging the sensor 26 with portions of their mouth or saliva in their mouth.

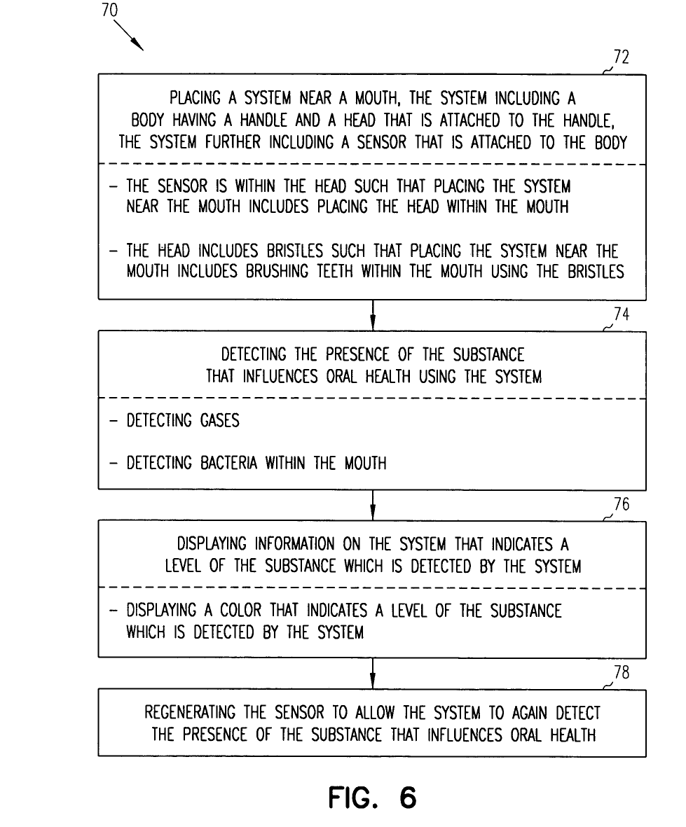

FIG. 6 illustrates an example method 70 of the present invention. The method 70 includes placing a system near a mouth 72 (e.g., system 10 shown in FIG. 1). The system includes a body having a handle and a head that is attached to the handle. The system further includes a sensor that is attached to the body. The method 70 further includes detecting the presence of a substance that influences oral health using the system 74. It should be noted that detecting the presence of the substance that influences oral health 74 may include detecting gases that are within (or exhaled from) the mouth and/or detecting bacteria that are within the mouth.

In some embodiments, the sensor may be within the head of the body such that placing the system near the mouth 72 may include placing the head within the mouth. In addition, the head may include bristles such that placing the system near the mouth 72 may include brushing teeth within the mouth using the bristles.

The method 70 may further include displaying information on the system that indicates a level of the substance which is detected by the system 76. Displaying information on the system 76 may include displaying a color that indicates the level of the substance which is detected by the system. In some embodiments, the displayed color may be compared to a chart to determine the level of the substance that is within the mouth.

The method 70 may further include regenerating the sensor to allow the system to again detect the presence of the substance that influences oral health 78. As discussed above with regard to sensor 26, the amount of time that it takes for the sensor to regenerate will depend in part on the level of the substance that was detected by sensor.

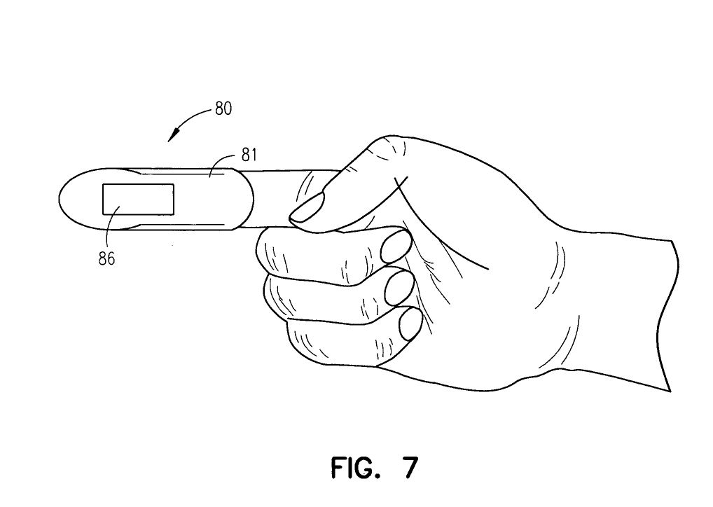

FIG. 7 illustrates another example system 80 of the present invention. The system 80 includes a finger glove 81 and a sensor 86 that is attached to the finger glove 81. The sensor 86 is adapted to detect substances that influence oral health when the system 80 is placed near (or in) a mouth.

It should be noted that finger glove 81 may be any type of finger glove that is known now or discovered in the future. Some example finger gloves are shown in U.S. Pat. Nos. 6,647,549 and 6,721,987.

In addition, the sensor 86 may be similar to any of the sensors 26 that are described or referenced above. The type of sensor 86 that is used in the system 80 will depend in part on the application where the system 80 is to be used.

A user operates the system 80 by manipulating the finger glove 81 within a mouth (e.g., over the teeth) to collect molecules that are related to oral malodor (e.g., hydrogen sulfide, dimethyl sulfide, methyl mercaptan, and ethyl mercaptan) and/or harmful bacteria (e.g., gingivitis bacteria). In some embodiments, sensor 86 may detect relative concentrations of these substances.

As discussed above with regard to sensor 26, sensor 86 may completely release the molecules that were collected during use such that the sensor 86 returns to an original state. The amount of time that it takes for the sensor 86 to regenerate will depend in part on the bacterial and/or chemical levels that are detected by the sensor 86.

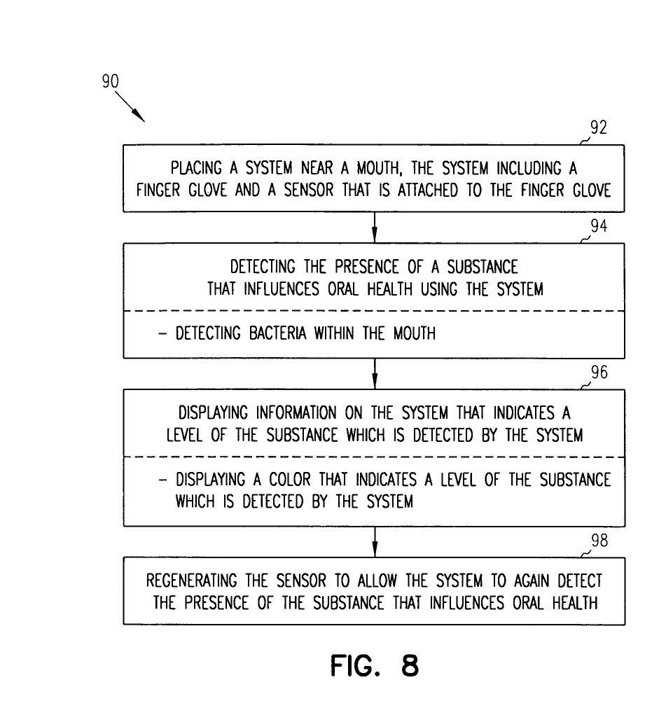

FIG. 8 illustrates another example method 90 of the present invention. The method 90 includes placing a system near a mouth 92 (e.g., system 80 in FIG. 7). The system includes a finger glove and a sensor that is attached to the finger glove.

The method 90 further includes detecting the presence of a substance that influences oral health using the system 94. It should be noted that detecting the presence of a substance that influences oral health 94 may include detecting gases and/or bacteria that are within (or near) the mouth.

The method 90 may further include displaying information on the system that indicates a level of the substance which is detected by the system 96. In some embodiments, displaying information on the system 96 may include displaying a color that indicates the level of the substance which is detected by the system.

The method 90 may further include regenerating the sensor to allow the system to again detect the presence of the substance that influences oral health 98. As discussed above with regard to sensors 26, 86, the amount of time that it takes for the sensor to regenerate will depend in part on the level of the substance that was detected by sensor.

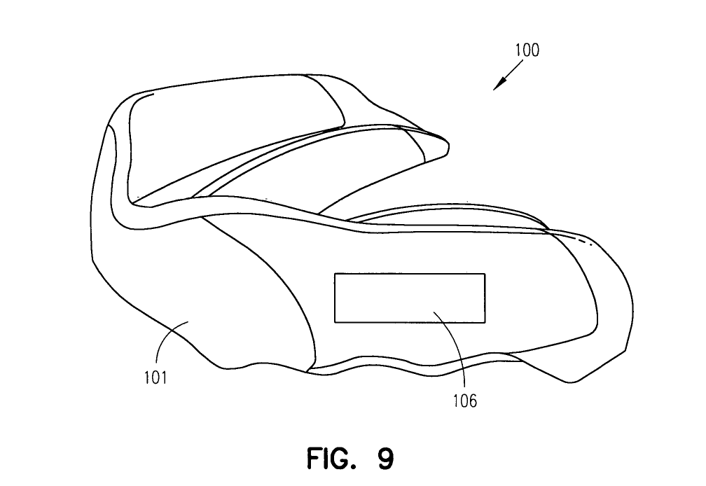

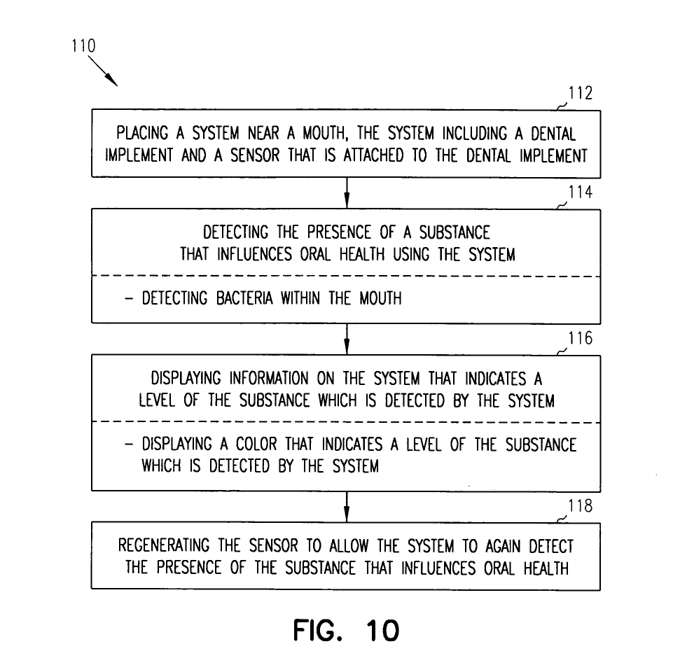

FIG. 9 illustrates another example system 100 of the present invention. The system 100 includes a dental implement 101 and a sensor 106 that is attached to the dental implement 101. The sensor 106 is adapted to detect substances that influence oral health when the system 100 is placed in a mouth.

It should be noted that dental implement 101 may be any type of dental implement that is known now or discovered in the future. In the example embodiment shown in FIG. 9, dental implement 101 is a mouthpiece that is adapted to be worn over the upper or lower teeth in a mouth. Some other example dental implements include retainers, dentures, bridges, crowns, tanners, tongue scrapers and toothpicks. In some embodiments, the dental implements may be part of head gear.

Sensor 106 may be similar to any of the sensors 26, 86 that are described or referenced above. The type of sensor 106 that is used the system 100 will depend in part on the application where the system 100 is to be used.

A user operates the system 100 by inserting the dental implement 101 within a mouth (e.g., over teeth) to collect molecules that are related to oral malodor (e.g., hydrogen sulfide, acetone, ammonia, dimethyl sulfide, methyl mercaptan, and ethyl mercaptan) and/or harmful bacteria (e.g., gingivitis bacteria).

As discussed above with regard to sensors 26, 86, sensor 106 may completely release the molecules that were collected during use such that the sensor 106 returns to an original state. The amount of time that it takes for the sensor 106 to regenerate will depend in part on the bacterial and/or chemical levels that were detected by sensor 106.

FIG. 10 illustrates another example method 110 of the present invention. The method 110 includes placing a system in a mouth 112 (e.g., system 100 shown in FIG. 9). The system includes a dental implement (e.g., a mouthpiece) and a sensor that is attached to the dental implement.

The method 110 further includes detecting the presence of a substance that influences oral health using the system 114. It should be noted that detecting the presence of a substance that influences oral health 114 may include detecting gases and/or bacteria that are within the mouth.

The method 110 may further include displaying information on the system that indicates a level of the substance which is detected by the system 116. In some embodiments, displaying information on the system 116 may include displaying a color that indicates the level of the substance which is detected by the system.

The method 110 may further include regenerating the sensor to allow the system to again detect the presence of the substance that influences oral health 118. As discussed above with regard to sensors 26, 86, 106, the amount of time that it takes for the sensor to regenerate will depend in part on the level of the substance that was detected by sensor.

It should be noted that any of the systems described herein may indicate the presence of harmful bacterial and/or oral malodor in the mouth. In addition, the systems may be configured to detect pH levels or other oral health parameters. Any of the systems that are shown and/or described herein may be used in any of the methods that are described or referenced herein. While the invention has been described in detail with respect to specific embodiments, it will be appreciated that there are variations of, and equivalents to these embodiments. Accordingly, the scope of the present invention should be determined by the appended claims and any equivalents thereto.

1. A system comprising:
a body that includes a handle and a head attached to the handle, the head being detachable from the handle; and
a sensor attached to the body, the sensor being adapted to detect a substance that influences oral health when the system is placed near a mouth.

2. The system of claim 1 wherein the sensor is within the handle.

3. The system of claim 1 wherein the sensor is within the head.

4. The system of claim 1 wherein the head includes bristles.

5. (canceled)

6. The system of claim 1 wherein the head is movable relative to the handle when the head is attached to the handle.

7. The system of claim 1 wherein the sensor is an electronic sensor.

8. The system of claim 1 wherein the sensor is chemically altered as the sensors sensor detects the substance that influences oral health.

9. The system of claim 8 wherein the sensor changes color as the sensor detects the substance that influences oral health.

10. The system of claim 1 wherein the sensor detects the substance that influences oral health when the system is placed within the mouth.

11. The system of claim 1 wherein the sensor detects gases.

12. The system of claim 1 wherein the sensor detects the substance within saliva.

13. A method comprising:
placing a system near a mouth, the system including a body having a handle and a head that is attached to the handle, the system further including a sensor that is attached to the body, wherein the head includes bristles such that placing the system near the mouth includes brushing teeth within the mouth using the bristles; and
detecting the presence of a substance that influences oral health using the system.

14-15. (canceled)

16. The method of claim 13 wherein detecting the presence of the substance that influences oral health includes detecting gases.

17. The method of claim 13 wherein detecting the presence of the substance that influences oral health includes detecting bacteria within the mouth.

18. The method of claim 13 further comprising displaying information on the system that indicates a level of the substance which is detected by the system.

19. The method of claim 18 wherein displaying information on the system includes displaying a color that indicates a level of the substance which is detected by the system.

20. The method of claim 13 further comprising regenerating the sensor to allow the system to again detect the presence of the substance that influences oral health.

21. A system comprising:
a finger glove; and
a sensor attached to the finger glove, the sensor being adapted to detect a substance that influences oral health when the finger glove is placed in a mouth.

22. The system of claim 21 wherein the sensor is an electronic sensor.

23. The system of claim 21 wherein the sensor is chemically altered as the sensors sensor detects the substance that influences oral health.

24. The system of claim 23 wherein the sensor changes color as the sensor detects the substance that influences oral health.

25. The system of claim 21 wherein the sensor detects the substance within saliva.

26. A method comprising:
placing a system near a mouth, the system including a finger glove and a sensor that is attached to the finger glove; and
detecting the presence of a substance that influences oral health using the system.

27. The method of claim 26 wherein detecting the presence of the substance that influences oral health includes detecting bacteria within the mouth.

28. The method of claim 26 further comprising displaying information on the system that indicates a level of the substance which is detected by the system.

29. The method of claim 28 where displaying information on the system includes displaying a color that indicates a level of the substance which is detected by the system.

30. The method of claim 26 further comprising regenerating the sensor to allow the system to again detect the presence of the substance that influences oral health.

31. A system comprising:
a dental implement; and
a sensor attached to the dental implement, the sensor being adapted to detect a substance that influences oral health when the system is placed within a mouth, wherein the sensor changes color as the sensor detects the substance that influences oral health.

32. The system of claim 31 wherein the dental implement is a mouthpiece.

33. The system of claim 31 wherein the sensor is an electronic sensor.

34. The system of claim 31 wherein the sensor is chemically altered as the sensor detects the substance that influences oral health.

35. (canceled)

36. A method comprising:
placing a system within a mouth, the system including a dental implement and a sensor that is attached to the dental implement such that placing the system within the mouth includes engaging the dental implement with teeth within the mouth; and
detecting the presence of a substance that influences oral health using the system.

37. The method of claim 36 wherein the dental implement is a mouthpiece such that placing the system within the mouth includes placing the mouthpiece over teeth within the mouth.

38. The method of claim 36 wherein detecting the presence of the substance that influences oral health includes detecting bacteria within the mouth.

39. The method of claim 36 further comprising displaying information on the system that indicates a level of the substance which is detected by the system.

40. The method of claim 36 further comprising regenerating the sensor to allow the system to again detect the presence of the substance that influences oral health.
